(19) United States
(12) Patent Application Publication
(10) Pub. No.: US 2011/0208081 A1
(43) Pub. Date: Aug. 25, 2011

(54) APPARATUS AND METHOD

(76) Inventors: Trevor . Smith, Maidstone (GB); Leigh Greenham, Watlington (GB); Philip Bromley, Rainham (GB)

(21) Appl. No.: 12/674,776

(22) PCT Filed: Sep. 8, 2008
(86) PCT No.: PCT/GB08/50794

§ 371 Date: May 27, 2010

(30) Foreign Application Priority Data
Sep. 7, 2007 (GB) 0717433.7

(51) Int. Cl. A61B 5/08

(52) U.S. Cl. 600/532

(57) ABSTRACT

A breath testing apparatus having a sampling device (10) and a sensor (50), wherein said sampling device is detachable. A method of analysing the breath of a patient is also provided.

### APPARATUS AND METHOD

The present invention relates to an apparatus capable of detecting exhaled gases in breath samples and the method of use of such apparatus. The present invention especially relates to apparatus and methods for detecting nitric oxide in exhaled breath.

The detection of compounds present in human breath in order to diagnose disease is known in the art. For example, the presence of volatile compounds such as acetone, ammonia or sulphur are well known for use in detecting conditions such as diabetes, liver impairment and kidney dysfunction.

Nitric oxide (NO) has been identified as a biomarker for airway inflammation, particularly associated with asthma. Bench-top apparatus for detecting NO in parts per billion (ppb) levels have been available since the 1990s. These were developed for use by clinicians to help assess appropriate treatment for asthmatics. Such equipment generally utilises the gas sensing principle of chemiluminescence.

Gas sensors utilising the principle of electrochemistry can be used but problems exist with the NO detectors currently available; problems range from their sensitivity to humidity present in the breath sample to be tested to the relatively slow response time of the NO sensors in the apparatus.

The latter problem has lead to the need for sample retention chambers and pumps in prior art devices to ensure a regulated flow of breath sample to the NO sensor to give meaningful results. The presence of pumps and retention chambers has meant that the size of the available equipment has remained relatively large and the costs relatively high.

NO can be used as a biomarker in several ways to give information about a patient. There are in principle three types of “breath” which can be measured for the presence of NO. Firstly, orally exhaled air (exhaled from the lower respiratory tract but which has not passed through the nasal passages), secondly nasally exhaled air (i.e. that which has passed though the nasal passages, preferably that which comes exclusively from the nasal passages), and thirdly regurgitated air from the stomach. These will all naturally contain different levels of NO and the increase or decrease of NO in the different types of breath act as indicators of medical conditions.

Firstly, levels of NO in orally exhaled air, also described as lower respiratory tract breath in the art, can indicate the presence of airway inflammation, typical experienced by asthmatics. Higher than normal levels of NO in orally exhaled breath is, for example, used as an indicator of asthma. The measurement of NO levels in orally exhaled breath is also useful in monitoring chronic obstructive pulmonary disease and interstitial lung disease.

Secondly, nasally exhaled air levels of NO are higher than levels of NO in orally exhaled air in healthy individuals. Elevated levels of NO in nasally exhaled air above the normal level can indicate the presence of allergic rhinitis. Lower levels of NO in nasally exhaled air is indicative of primary ciliary dyskinesia, and extremely low levels of nasal NO have been reported in cystic fibrosis.

The third type of NO containing “breath” is that regurgitated from the stomach. NO levels in regurgitated air can be correlated with gastric disturbances, and a lower level of NO in the regurgitated air has been found to correlate with lower levels of stomach HCl, and so can be indicative of gastro-oesophagal reflux.

Where orally exhaled air is to be measured, it is important to ensure that the air has been inhaled via the mouth and not the though the nasal passages. Where this is not ensured, the level of NO in the exhaled breath will be appear higher as it will contain endogenous nasal NO in the sample.

The present invention is concerned with improved sampling devices for measuring levels of gases in exhaled breath, especially but not exclusively orally exhaled breath. The invention is particularly suited to the detection of NO in humans, but may be used for the detection of other gases of diagnostic value as will become apparent. Furthermore, the invention may also be used for detecting nitric oxide and other gases of diagnostic value in the exhaled breath of animals in a veterinary application.

According to the present invention there is provided a breath testing apparatus comprising a sampling device and a sensor device to detect levels of a gas of interest in exhaled air, wherein said sampling device is detachable.

The sensor device and the sampling device each comprise corresponding interface means to allow the devices to be connected together. Advantageously the connection between the sampling device and the sensor device is substantially airtight.

In a preferred embodiment of the invention, the connection comprises a plug and socket arrangement. It is preferred that the interface means on the sensor device comprises a recess into which the interface means on the sampling device is inserted. Retaining means to hold the devices together, such as a clip, may be provided. However, the interface means are desirably formed such the friction between the two means is sufficient to hold the devices together for use. It is preferred that the interface means are shaped such that they can fit together only in one orientation; a D-shaped means is especially preferred.

The sampling device comprises a patient contact means. The patient contact means suitably comprises a mouthpiece, a facemask, a nasal breath sampling means or a combination of one or more of these. A mouthpiece is preferred as it is simple to use.

The sampling device typically comprises a conduit running between the patient contact means and the interface means.

It is desirable that the sampling device comprises infection control means to prevent infectious particles from passing through the sampling device into the sensor device. Such infection control means may suitably comprise a filter which is able to remove particles as small as viruses, bacteria and other potentially infective microbes. Such filters are well known in the art. The infection control means is suitably positioned in the conduit between the patient contact means and the interface means. Where the sampling device comprises such an infection control means, it is a significant advantage that the sensor device can be reused without the need for sterilisation between patients. The present invention thus provides that the two parts of the breath sample test apparatus are separable from one another, such that the sampling device can be removed and/or replaced. This is advantageous as the sampling device, which is the point of contact for the patient can be disposed of after use and replaced with a new clean/sterile sampling device. In this way, the sampling device may be a single use, disposable unit.

In a preferred embodiment of the present invention, the sensor device comprises a sensor for detecting the level of a gas of interest within the exhaled air. Preferably, the sensor device further comprises a pump configured to extract a portion of the exhaled air for detection by the sensor. In this embodiment, the extracted portion may be drawn through a solenoid valve to be brought into contact with the sensor, into which it diffuses.

The sensor of the sensor device is preferably adapted to detect levels of nitric oxide (NO) within the exhaled air, but may of course be suitable for any gas of diagnostic value. Preferably the detector is able to detect NO down to ppb levels.

In a preferred embodiment of the present invention the sensor device is adapted such that the sensor can be removed and replaced with a sensor for a different gas of interest. Other gases which may be of interest include carbon monoxide or hydrogen. This allows one sensor device to be used for a number of different sensing operations.

Typically the sensor is an electrochemical gas sensor. It is particularly advantageous to use a sensor that is temperature stable, this negates the need for heating or cooling of the sensor or exhaled air during use. This offers space saving opportunities in the apparatus design and simplicity of design and construction of the apparatus.

Preferably the sensor device contains an independent power source, such as a battery, such that it is portable and can be used away from the power grid. Preferably the sensor device contains a back-up battery to ensure power to a memory means within the sensor device which contains software and/or to the gas sensor.

It is envisaged that the breath testing apparatus of the present invention will be shaped and sized such that it is suitable for hand-held operation; this advantageously allows the unit to be used in a domestic setting, without a trip to a trained clinician being necessary. This would allow suffers of, e.g. asthma, to assess their NO levels in the home and aid in their ongoing treatment and monitoring. Additionally, smaller apparatus for testing NO levels provides general space saving benefits.

The apparatus of the present invention advantageously negates the need to have an additional breath storage chamber and a pumping device to ensure a controlled flow rate to the sensor. This further facilitates a small and simple breath sample testing apparatus.

The sampling device preferably comprises a flow regulator. The flow regulator ensures that exhaled air reaches the gas sensor in a controlled manner.

The flow regulator may take the form of a mechanical device to restrict or otherwise actively regulate the flow of exhaled air. Such mechanical devices are known in the art.

However, a preferred flow regulator is a flow indicator which indicates to a patient the rate of flow and allows the patient to adjust the rate of exhalation accordingly. Conveniently the flow indicator comprises indication means to indicate to the patient that a desired flow rate or range of flow rates is being achieved. Such indication means may be visual or aural. Suitably the indicator means is a visual indicator, such as a scale with the desired flow rate indicated thereupon. The flow indicator preferably comprises a body which is adapted to interact with the flow of breath and have its position or orientation influenced according to the flow rate. Suitably the body is located within a conduit through which at least a portion of the flow of exhaled air will pass, and is moved within the conduit depending on the rate of flow. The conduit in which the body is located is suitably part of the conduit running from the patient contact means to the interface means. The conduit may suitably be arranged such that it is vertical during use and, as such, the body is pushed against the force of gravity by the flow of exhaled air; the height thus depending on the flow rate. Alternatively the body may move against the action of a resilient means, such as a spring for example.

Suitably the body is a ball or bead, preferably having a substantially spherical shape.

It will be obvious that the body must not completely obstruct the conduit such that air can flow around the body. Suitably the conduit comprises retaining means to ensure the body remains located within a desired region of the conduit. The retaining means may be one or more narrowings of the conduit to a dimension smaller than the diameter of the body.

Suitably the conduit has a suitable profile such that as the body moves further along the conduit, air is able to flow more easily around the body; for example, a tapered profile.

Preferably the flow regulator is adapted to provide a flow rate of from 30 to 70 ml/s, especially from 45 to 55 ml/s (45 to 55 ml/s is recommended by the American Thoracic Society and The European Respiratory Society (ATS/ERS) recommendations for measuring exhaled NO levels).

Suitably the sampling device comprises an inlet to allow the patient to inhale air through the sampling device. In one embodiment the inlet is provided on the conduit. In an alternative embodiment the inlet is the interface means of the sampling device, in which case the air being inhaled passes through the sensor device prior to entering the sampling device. Suitably the apparatus comprises an inhalation filter to filter the air being inhaled through the sampling device; the filter can be provided as part of the sensor device or the sampling device depending on the arrangement of the inlet. Where the apparatus is intended to detect levels of NO within the exhaled air, the inhalation filter is suitably adapted to remove NO from the air being inhaled. Suitable filters for removing NO include a potassium permanganateKMnO₄ filter and/or carbon beads/carbon material filter. The filter is also desirable configured to remove any particulates or other component parts of the ambient air which could adversely affect the operation and/or accuracy of the sensor device.

In a preferred embodiment the inlet is the interface means, and the air to be inhaled passes through an opening in the sensor device which is provided with a NO filter. The air to be inhaled can conveniently pass though the sensor, which can then be calibrated to “zero” on the NO free air being drawn in. This provides a convenient means of self calibration. The air then passes through the interface means into the sampling device and is inhaled by the patient.

It is desirable that the sampling device comprises one or more one-way valve to direct the paths of the inhaled and exhaled air. Preferably, the one or more one-way valve allows air to pass to the patient during an inhalation phase, but closes, preventing air passing out of the inlet, or otherwise by-passing the flow regulator or outlet, during an exhalation phase. Such one-way valves are well known in the art and, in one embodiment, may comprise a simple flap and aperture arrangement.

In the inlet arrangement described above, where the inlet is the interface means, the one-way valve is adapted to allow air being inhaled to bypass the flow regulator when being inhaled, but which closes during the exhalation phase to force the air to pass through the flow regulator.

The sampling device desirably comprises a means to remove water from the exhaled breath of the patient. Suitably this comprises a humidity filter. The humidity filter may suitably be located in the conduit of the sampling device between the patient contact means and the interface means. The humidity filter removes water from the exhaled air of the patient. With many sensor devices it is essential that the air does not contain significant levels of water as it can interfere with the result and/or damage the sensor itself. In a particularly preferred embodiment of the invention, it is beneficial that the humidity filter reduces the humidity level of the exhaled breath to a pre-determined level rather than zero.

Conveniently the infection control means and the means to remove water can be provided by a multi-function filter.

The sampling device is suitably formed substantially from a plastics material. Conveniently the sampling device may be moulded. Suitably the plastics material is impregnated with an antimicrobial agent.

The sampling device may comprise a flow restriction means which provides sufficient resistance to exhalation such that the nasal vellum of the patient is closed during exhalation, and thus nasally exhaled air is substantially excluded from the tested breath.

The electronics and software required to control and operate a breath testing apparatus of the present invention are known in the art.

In a further aspect the present invention relates to a method of analysing the breath of a patient for the presence or amount of a gas of interest, wherein the patient exhales into a breath sample test apparatus as discussed above.

In a preferred embodiment the method is a method of analysing the NO content of the exhaled air of the patient.

Suitably the method comprises the steps of:
providing a breath testing apparatus as set out above;
causing the patient to exhale into the apparatus at a suitable rate; and
analysing the exhaled breath for the presence or amount of a gas of interest.

The method suitably includes the step of providing to the user an indication of the rate of exhalation so that they can alter the exhalation rate such that it falls within a desired range. This may conveniently be achieved using a floating ball or similar arrangement described above. Suitably the rate of exhalation is between 45 and 55 ml/s for a time sufficient for sampling to occur. Most preferably, the rate of exhalation is 50 ml/s.

Preferably, the method further comprises the step of enabling the patient to inhale through the breath testing apparatus.

Suitably, the method comprises the step of extracting a portion of the exhaled air for analysis within the sensor device of the breath testing apparatus. This step may involve the use of a pump contained in the sensor device to effect extraction.

The method may comprise the step of comparing the result of the method with an expected value. From this a diagnostic or prognostic indication may be derived.

The method may comprise the step of disposing of the sampling device of the apparatus after use. A new sampling device can be connected to the sensor device prior to another sample being analysed.

Suitably the method complies with ATS/ERS recommendations.

In a further aspect the present inventions provides a sampling device as discussed above. The sampling device may be disposable item and thus may be supplied separately from the apparatus.

Embodiments of the present invention will now be described, by way of non-limiting example only, with reference to the accompanying drawings in which;

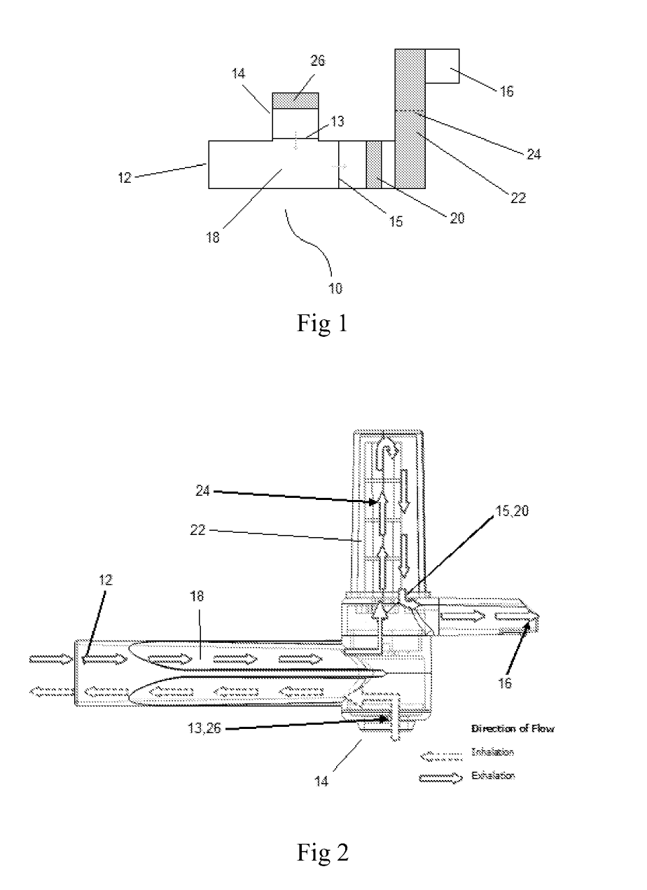

FIG. 1 shows a schematic view of a sampling device in accordance with the present invention;

FIG. 2 shows a more detailed cross section of a sampling device in accordance with the present invention;

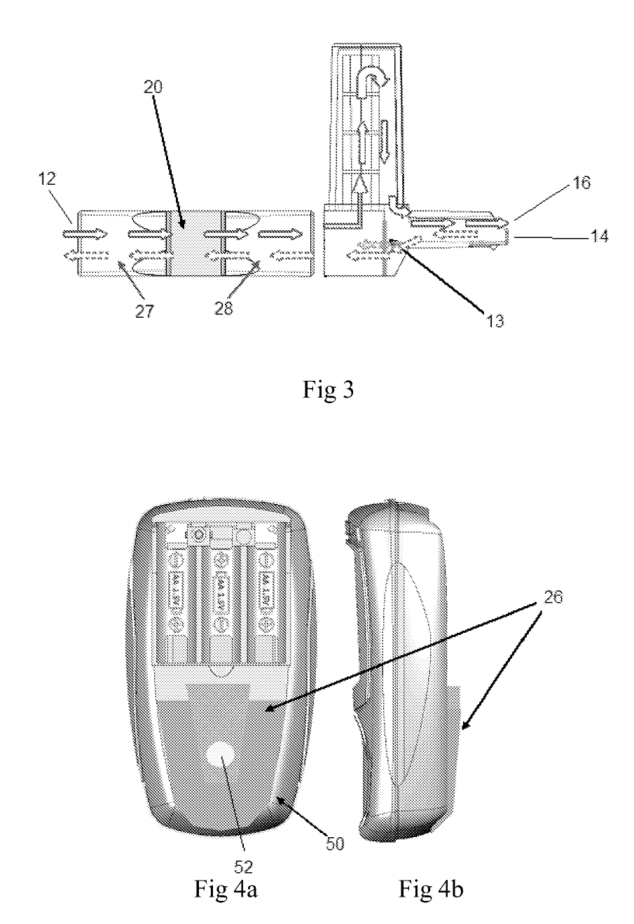

FIG. 3 shows a schematic view of an alternative sampling device;

FIG. 4a shows a rear view of a sensor device; and

FIG. 4b shows a side view of a sensor device.

A breath test apparatus comprises a sampling device 10 and a sensor device 50. The sensor device 50 and the sampling device 10 connect together via cooperating interface means 16. The interface means 16 of the sensor device suitably comprise a conduit of D-shaped profile on the sampling device 10, which may be inserted into a correspondingly shaped D-shaped recess on the sensor device 50. The interface means 16 is suitably tapered to ensure a neat fit (not shown). The connection formed is preferably substantially air tight.

The sampling device 10 comprises a patient contact means 12 which is suitable for a patient to breath into. The patient contact means 12 comprises a mouthpiece, which is shaped and sized such that a patient can put the device into their mouth and for a seal with their lips. A pipe which is circular or elliptical in profile is suitable.

The patient contact means 12 is connected to a conduit 18. The conduit 18 is provided with a first one way valve 15 which allows exhaled breath to pass through, but which does not allow any return of air therethrough.

An inlet 14 is provided which leads into the conduit 18 at a point between the patient contact means 12 and the first one way valve. The inlet 14 is provided with a second one way valve 13 which is adapted such that atmospheric air can pass through the valve 13 into the conduit 18 when the patient inhales, but prevents air escaping from the inlet 14 during an exhalation phase. The direction of flow through the one way valves is shown by arrows in FIG. 1.

As a result of the arrangement of valves and conduits, when the patient inhales, air passes through the inlet 14 into the conduit 18, and through the patient contact means 12 and into the patient's airways. Upon exhalation the air passes into the conduit 18 and then though the first one way valve 15 and through the remainder of the sampling device 10.

In an alternative arrangement, as shown in FIG. 3, the sampling device comprises a single one way valve which allows air by-pass the flow regulator as it is inhaled, but which closes during exhalation to force air to pass through the flow regulator during exhalation.

In one embodiment the inlet 14 comprises a filter 26 which is capable of removing NO from the air passing into the conduit 18. A suitable filter material is KMnO₄ and/or carbon granules. The filter 26 is positioned such that air passes through the filter 26 before passing through the second one way valve 13. This filter ensures that air inhaled by the patient is substantially free from NO.

Alternatively, as shown in FIGS. 3 and 4, the NO filter 26 can be provided on the sensor device 50 such that air is drawn through an opening 52 on the sensor device 50 and thereafter into the sampling device 10 through the interface means 16. This arrangement has the benefit that NO air passing through the sensor device during inhalation can be used to “zero” the sensor. In this case the inlet 14 of the sampling device 10 is the interface means 16—the air ultimately being drawn in through the opening 52 on the sensor device 50.

The conduit 18 is provided with a filter 20 which is capable of both removing water from the exhaled air and filtering out infections particles. The filter 20 may comprise two separate filters, one for water, the other for small particles. Suitable filters for each of these purposes are well known in the art. This filter 20 ensures that exhaled air from the patient is dried and cleaned from potentially infectious particles before it passes through the sampling device 10 and eventually into the sensor device 50.

The conduit 18 then becomes a flow indicator conduit 22 which, in use, is orientated vertically. The flow indicator conduit 22 is transparent, and has an indicator 24 marked upon it. The indicator 24 is a scale which indicates the rate of flow. The indicator conduit 22 contains a ball (not shown) which is of smaller diameter than the conduit 22. When the patient exhales through the sampling device 10, air passing through the indicator conduit 22 causes the ball to rise and it is pushed up the conduit 22. The height the ball rises is relative to the flow of air through the conduit 22. The conduit 22 is configured such that as the ball rises within the conduit 22 the exhaled air is able to pass more easily around the ball; this is easily achieved with a tapering internal diameter of the conduit 22. The indicator 24 shows a preferred region within which the ball should be maintained, this region corresponding to a flow of 45 to 55 ml/s.

The indicator conduit is arranged such that it will be in the line of sight of a patient exhaling into the device.

The breath test apparatus as set out above will generally provide enough resistance to exhalation to close the patients nasal vellum during use. If additional resistance is required a constriction of one of the conduits or other parts of the sampling device through which exhaled air passes may be provided.

In one preferred embodiment shown in FIG. 3, the conduit 18 may comprise two section 27, 28 which surround a section comprising the filter 20. The sections 27, 28 can conveniently be identical sections adapted to interface with the filter at one end, the other end being adapted to either interface with the rest of the sampling device 10, or to provide or interface with the user contact means 12.

The sensor device 50 comprises is shaped and sized such that it is holdable in the hand of a user.

The sensor device 50 comprises an electrochemical gas sensor which is mounted within the sensor device such that exhaled air passing through the interface means 16 passes over the relevant portion of the gas sensor before exiting the device through an exhaust port 52. The exhaust port 52 can conveniently be the same as the opening 52 which allows air to be inhaled through the sensor device 50. Suitably the gas sensor is arranged such that a gas entry surface faces into a diffusion cavity through which the exhaled air passes.

The gas sensor may be essentially any gas sensor, though sensors for NO, CO and H₂ are of particular interest.

The electrochemical gas sensor is mounted in the sensor device 50 such that it is removable and may be replaced as required. This allows the sensor to be replaced if it is not performing as desired, or it may be replaced with a sensor for another type of gas. When the gas sensor is changed to a sensor for a different type of gas it is clearly essential that the electronics and software within the sensor device are adapted to the change of sensor. However, this is not difficult to achieve, and may simply involve switching between a number of different software packages, which are conventional for each type of sensor. Desirably the sensor device 50 automatically detects the type of gas sensor and adapts accordingly. This is conveniently achieved by providing each sensor with a suitable electronic indicator.

Suitable gas sensors are well known in the art. In particular, suitable electrochemical gas sensors are available that respond specifically to ppb levels of nitric oxide.

The gas sensor is provided with a battery back-up to keep the sensor biased as necessary, even when the device is switched off.

With electrochemical gas sensors it is generally not necessary to heat the diffusion cavity, the breath sample or the sensor, as they are temperature stable.

The sensor device 50 comprises a user interface. Suitably the user interface comprises a display, and input means to allow a user to operate the device. A touch sensitive LCD display is a desirable system which combines both input and display functions. The display, amongst other functions, indicates the amount of gas of interest in a sample.

In use a sampling device 10 is connected to the sensor device 50 to form the complete breath test apparatus. The sensor device 50 is then switched on and the gas sensor allowed sufficient time to become fully functional—this may be around 1 minute with current gas sensors.

The patient puts the mouthpiece in their mouth and breathes in (preferably through their mouth only—no air being breathed in through the nose). The air passing into their airways is scrubbed of NO as it enters the apparatus. The NO scrubbed air can conveniently be used to calibrate the gas sensor. The patient then exhales into the sampling device 10 at a slow and steady rate. The patient adjusts the rate of exhalation such that the ball is in the correct region of the indicator conduit 22.

The air passes through the humidity and infection control means before passing into the sensor device 50. A portion of the exhaled air is extracted by a pump (not shown) contained within the sensor device 50, which is drawn through a solenoid valve (not shown). This extracted sample comes into contact with the gas sensor and diffuses into it. The gas sensor detects the level of the gas of interest in the sample and an output is produced in the display.

The output from the gas sensor will increase in direct proportion to the concentration of gas, e.g. NO in the sensor cavity. This output is amplified and fed to a microcontroller on a PCB, where it is first digitised before being processed by embedded software. Mathematical algorithms within the software create a 3-second running average of the rising sensor output, compensating for temperature and humidity effects by virtue of monitoring said parameters within the sensor device 50. When this averaged value reaches a peak and starts to subside, the software calculates the equivalent ppb concentration of this peak and displays it on the display. By this method, the value shown will represent the so-called “plateau concentration” of NO, as required by the ATS/ERS recommendations.

One particular advantage of the present invention is that by housing all of the breath sampling parts necessary for a particular gas measurement, e.g. NO measurement, within a disposable sampling device, the sampling device instrument itself can be used for other applications that do not require such breath sampling parts. For example, when fitted with gas sensors for hydrogen or carbon monoxide (rather than NO), the instrument can be used for conventional breath analysis applications for gastric investigations or smoking cessation programmes.

1. A breath testing apparatus comprising:
a sampling device;
a sensor device to detect levels of a gas of interest in exhaled air; and
wherein said sampling device is detachable.

2. The breath testing apparatus of claim 1, wherein said sensor device and said sampling device each comprise corresponding interface means to allow said sensor device and said sampling device to be connected together.

3. The breath testing apparatus of claim 2, wherein a connection between said sampling device and said sensor device is substantially airtight.

4. The breath testing apparatus of claim 3, wherein said connection comprises a plug and socket arrangement.

5. The breath testing apparatus of claim 4, wherein said interface means are D-shaped.

6. The breath testing apparatus of claim 1, wherein said sampling device comprises a patient contact means.

7. The breath testing apparatus of claim 6, wherein said sampling device further comprises a conduit running between said patient contact means and said interface means.

8. The breath testing apparatus of claim 1, wherein said sampling device comprises infection control means.

9. The breath testing apparatus of claim 8, wherein said infection control means is positioned within said conduit.

10. The breath testing apparatus of claim 1, wherein said sensor device comprises a sensor for detecting a level of said gas of interest within said exhaled air.

11. The breath testing apparatus of claim 10, wherein the sensor device further comprises a pump configured to extract a portion of the exhaled air for detection by the sensor.

12. The breath testing apparatus of claim 10, wherein said sensor detects the levels of nitric oxide within said exhaled air.

13. The breath testing apparatus of claim 10, wherein said sensor device is adapted such that said sensor can be removed and replaced with a sensor for a different gas of interest.

14. The breath testing apparatus of claim 10, wherein said sensor is an electrochemical gas sensor.

15. The breath testing apparatus of claim 10, wherein said sensor is temperature stable.

16. The breath testing apparatus of claim 1, wherein said sensor device comprises an independent power source.

17. The breath testing apparatus of claim 16, wherein said sensor device further comprises a back-up battery to ensure power to a memory means within said sensor device.

18. The breath testing apparatus of claim 1, wherein said sampling device comprises a flow regulator.

19. The breath testing apparatus of claim 18, wherein said flow regulator comprises a flow indicator having a visual indication means.

20. The breath testing apparatus of claim 19, wherein said flow indicator further comprises a body which is adapted to interact with the flow of breath and have its position or orientation influenced according to a flow rate.

21. The breath testing apparatus of claim 18, wherein said flow regulator is adapted to provide a flow rate of from 30 to 70 ml/s.

22. The breath testing apparatus of claim 1, wherein said sampling device further comprises an inlet to allow a patient to inhale air through the sampling device.

23. The breath testing apparatus of claim 22, wherein said breath testing apparatus comprises an inhalation filter to filter the air being inhaled through said sampling device.

24. The breath testing apparatus of claim 22, wherein said sampling device comprises at least one-way valve to direct paths of the inhaled and exhaled breath.

25. The breath testing apparatus of claim 24, wherein said sampling device comprises a means to remove water from the exhaled breath of the patient.

26. The breath testing apparatus of claim 25, wherein said means comprises a humidity filter.

27. The breath testing apparatus of claim 26, wherein said humidity filter is located in a conduit of said sampling device.

28. The breath testing apparatus of claim 26, wherein said humidity filter reduces the humidity level of said exhaled breath to a pre-determined level.

29. The breath testing apparatus of claim 8, wherein said infection control means and a means to remove water are provided by a multi-function filter.

30. The breath testing apparatus of claim 1, wherein said sampling device comprises a flow restriction means which provides sufficient resistance to exhalation such that a nasal vellum of a patient is closed and nasally exhaled air is substantially excluded from a tested breath.

31. The breath testing apparatus of claim 1, wherein said sampling device is formed substantially from a plastics material.

32. The breath testing apparatus of claim 31, wherein said plastics material is impregnated with an antimicrobial agent.

33. The breath testing apparatus of claim 1, wherein said breath testing apparatus is shaped and sized such that it is suitable for hand-held operation.

34. The breath testing apparatus of claim 1, wherein the sampling device is a single use, disposable unit.

35. A method of analyzing breath of a patient for the presence or amount of a gas of interest:
providing a breath testing apparatus;
causing the patient to exhale into said breath testing apparatus at a suitable rate; and
analyzing the exhaled breath for the presence or amount of the gas of interest.

36. The method of claim 35 comprising providing to a user an indication of the rate of exhalation so that they can alter the exhalation rate such that it falls within a desired range.

37. The method of claim 35 comprising enabling the patient to inhale through said breath testing apparatus.

38. The method of claim 35 comprising extracting a portion of the exhaled air for analysis within a said sensor device of said breath testing apparatus.

39. The method of claim 35 comprising comparing a result of said method with an expected value.

40. The method of claim 35 comprising disposing a sampling device of said breath testing apparatus after use.

41. The method of claim 35 comprising analyzing a nitric oxide content of the exhaled air of the patient.
